(19) United States
(12) Patent Application Publication
(10) Pub. No.: US 2010/0204074 A1
(43) Pub. Date: Aug. 12, 2010

(54) Compounds That Are Liquid At Ambient Temperature

(75) Inventors: Hendrik Huesken, Haltern am See (DE); Paul Birnbrich, Solingen (DE); Gilbert Schenker, Erkrath (DE)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Dusseldorf (DE)

(21) Appl. No.: 12/281,610

(22) PCT Filed: Feb. 23, 2007
(86) PCT No.: PCT/EP2007/001572

§ 371 Date: Sep. 3, 2008

(30) Foreign Application Priority Data
Mar. 3, 2006 (DE) 102006009973.7

(51) Int. Cl. B01D 11/04; C07C 211/03; C07C 211/00; C07F 9/02; B01F 1/00; B01J 31/00; C09K 3/00; C10M 141/10; C10M 141/06; C09K 5/00; A61K 8/40; A61K 8/30; B01D 53/14

(52) U.S. Cl. 508/421; 564/463; 564/305; 568/9; 252/364; 502/164; 252/184; 508/547; 252/78.5; 252/77; 512/1; 210/634; 95/149

(57) ABSTRACT

Disclosed are ionic liquids that have a low melting point. Said liquids are provided in the form of compounds that are selected among the group comprising tetraalkyl ammonium salts, benzyl trialkyl ammonium salts, tetraalkyl phosphonium salts, and benzyl trialkyl phosphonium salts, provided that at last two alkyl groups represent aliphatic radicals with chain lengths ranging from 8 to 10 C atoms, and provided that not all alkyl radicals are identical.

### CROSS REFERENCE TO RELATED APPLICATIONS

This application is the National Phase entry of PCT/EP2007/001572, filed Feb. 23, 2007, which claims priority to German patent application number DE 102006009973.7, filed Mar. 3, 2006, both of which are incorporated herein by reference in their entireties.

### FIELD OF THE INVENTION

This invention relates to ionic fluids having a low melting point.

### BACKGROUND OF THE INVENTION

Ionic fluids are generally understood to be organic salts, or mixtures thereof, which have a melting point below 100° C. (cf. the review article by P. Wasserscheid and W. Keim in Angew. Chem. 2001 (112), pp. 3773-3789). These salts typically consist of anions such as, for example, halides, halostannates, haloaluminates, hexafluorophosphates or tetrafluoroborates combined with substituted ammonium, phosphonium, pyridinium, triazolium, pyrazolium or imidazolium cations.

### BRIEF SUMMARY OF THE INVENTION

Ionic fluids have very recently become highly topical. An important application is their use as solvents for organic substances. There is a constant need here for substances which have as low a melting point as possible.

The problem addressed by the present invention was first and foremost to provide new ionic fluids having a melting point of at most ambient temperature (a temperature of 30° C. in the context of the present invention). More particularly, the problem addressed by the present invention was to lower the melting point of ionic fluids of the tetra-alkyl ammonium salt type, the tetra-alkyl phosphonium salt type and the like.

According to the invention, the problem stated above is solved by compounds selected from the group consisting of
tetra-alkyl ammonium salts,
benzyl trialkyl ammonium salts,
tetra-alkyl phosphonium salts and
benzyl trialkyl phosphonium salts,
   with the proviso that at least two alkyl groups are aliphatic radicals having chain lengths of 8 to 10 carbon atoms.

### DETAILED DESCRIPTION OF THE INVENTION

Accordingly, the present invention relates to compounds liquid at ambient temperature selected from the group consisting of tetra-alkyl ammonium salts, benzyl trialkyl ammonium salts, tetra-alkyl phosphonium salts and benzyl trialkyl phosphonium salts, with the proviso that at least two alkyl groups are aliphatic radicals having chain lengths of 8 to 10 carbon atoms and with the additional proviso that the alkyl groups should not all be identical.

Basically, the present invention is not limited in regard to the nature of the anions. For example, halides (CI, Br, I), halostannates, haloaluminates, hexafluorophosphates, tetrafluoroborates, tetrahydridoborates, sulfates, alkyl sulfates, hydrogen sulfates and carbonates may be used as anions. However, halides (chloride, bromide, iodide) or alkyl sulfates, more particularly chloride, are preferably used as anions.

The compounds according to the invention, which are ionic fluids, have melting points below 30° C. In this connection, a comparison with the melting points of tetra-alkyl ammonium halides having identical alkyl groups is revealing:

| | |
|---|---|
| tetrabutyl ammonium bromide | 100-103° C. |
| tetrabutyl ammonium hydrogen sulfate | 169-172° C. |
| tetrabutyl ammonium tetrahydridoborate | 124-129° C. |
| tetrabutyl ammonium iodide | 143-146° C. |
| tetrapentyl ammonium iodide | 135-137° C. |
| tetrahexyl ammonium iodide | 121-124° C. |
| tetra-octyl ammonium bromide | 95-98° C. |

All these compounds have extremely high melting points. Accordingly, it was surprising that the compounds according to the present invention, more particularly the compounds of formula (I), are distinguished by the requisite low melting points.

In a preferred embodiment, the present invention relates to compounds liquid at ambient temperature corresponding to formula (I):

[NR¹R²R³R⁴]+[X]— (I)

where the substituent R¹ is a benzyl group or an alkyl group containing 1 to 4 carbon atoms and the substituents R² to R⁴ independently of one another are alkyl groups containing 8 to 10 carbon atoms and X is a halide anion or an alkyl sulfate anion with the formula [R⁵SO₄]⁻, where R⁵ is an alkyl group containing 1 to 12 carbon atoms, with the proviso that the substituents R² to R⁴ should not all be identical. In one embodiment, X is a chloride anion. In another embodiment, X is an anion selected from the group consisting of 2-ethyl hexyl sulfate, octyl sulfate and decyl sulfate.

In a particularly preferred embodiment, the present invention relates to compounds liquid at ambient temperature corresponding to formula (I-a):

[NCH₃R²R³R⁴]+[X]— (I-a)

where the substituents R² to R⁴ independently of one another are alkyl groups containing 8 to 10 carbon atoms and X is a halide anion or an alkyl sulfate anion with the formula [R⁵SO₄]⁻, where R⁵ is an alkyl group containing 1 to 12 carbon atoms, with the proviso that the substituents R² to R⁴ should not all be identical.

The compounds I and, more particularly, I-a may be used individually or in admixture with one another. Basically, any methods known to the relevant expert may be used for their synthesis. More particularly, they are produced by subjecting trialkylamines corresponding to formula (II):

NR²R³R⁴ (II)

where the substituents R² to R⁴ independently of one another represent C₈₋₁₀ alkyl groups,
to a quaternization reaction with compounds R¹—X, where X is halogen. Information on the production of compounds I and I-a containing alkyl sulfate anions can be found, for example, in WO-A-03/02281.

In a preferred embodiment, the ionic fluid used is a mixture of compounds I-a containing chloride anions which has an average molecular weight (what is meant here is the number average molecular weight (Mn) known to the expert which is defined as follows: Mn=ΣnᵢMᵢ/Σnᵢ) in the range from 410 to 450, more particularly in the range from 420 to 440 and preferably in the range from 425 to 435. These mixtures are hydrophobic substances liquid at ambient temperature which may be used as solvents for any organic reactions.

The present invention also relates to compositions containing—based on the composition as a whole—(a) at least 85% by weight of one or more compounds corresponding to formula (I):

[NR¹R²R³R⁴]+[X]— (I)

where the substituent R¹ is a benzyl group or an alkyl group containing 1 to 4 carbon atoms and the substituents R² to R⁴ independently of one another are alkyl groups containing 8 to 10 carbon atoms and X is a halide anion or an alkyl sulfate anion with the formula [R⁵SO₄]⁻, where R⁵ is an alkyl group containing 1 to 12 carbon atoms,
and (b) 0.01 to 10% by weight water, with the proviso that the upper limit to the quantity of water is determined by the maximum solubility of water in the compounds I at 20° C.

In a preferred embodiment, the compositions used contain (a) at least 85% by weight of one or more compounds corresponding to formula I-a:

[NCH₃R²R³R⁴]+[X]— (I-a)

where the substituents R² to R⁴ independently of one another are alkyl groups containing 8 to 10 carbon atoms and X is a halide anion or an alkyl sulfate anion with the formula [R⁵SO₄]⁻, where R⁵ is an alkyl group containing 1 to 12 carbon atoms,
and (b) 0.01 to 5% by weight water, with the further proviso that, if the substituents R² to R⁴ are identical, at least two different compounds I-a (more particularly mixtures of the two species methyl tri-n-octyl ammonium halide and methyl tri-n-decyl ammonium halide) are used; the counter-ion of the compounds I-a is, in particular, chloride. To produce these compounds by the above-mentioned quaternization of trialkylamines II with methyl chloride, it is of particular advantage to use trialkylamines II of which the substituents R² to R⁴ are of native origin and emanate from C₈₋₁₀ fatty alcohols (which means that these trialkylamines are obtained from fatty alcohols which by far predominantly contain octanol and/or decanol, for example the corresponding Lorol types commercially obtainable from Cognis). The compositions mentioned are distinguished by very low melting points, in some cases even below 0° C.

It is expressly pointed out that the compositions mentioned represent a single homogeneous phase and are hydrophobic. Their hydrophobicity is reflected in their high solubility in organic solvents. The compositions mentioned are almost completely soluble in organic solvents, such as benzene, chloroform or isopropanol, at 20° C.

The above-described compounds of formula I or la, mixtures thereof with one another and the described compositions containing such substances and small amounts of water may be classed as ionic fluids. These ionic fluids may be used for a variety of applications, for example as solvents, solvent additives, phase transfer catalysts, extractants, lubricants, gas absorbing agents, solvents for batteries, fixateurs for perfumes or heat-exchange media. The present invention also relates to a chemical reaction in which an ionic fluid according to the present invention is used as a solvent, solvent additive or phase transfer catalyst; to electrochemical processes (for example electrolysis, electrocoating, etc.) in which an ionic fluid according to the invention is used as a solvent; to separation processes in which an ionic fluid according to the present invention is used as a solvent or solvent additive; to a heat-exchange apparatus in which an ionic fluid according to the invention is used as a heat-exchange medium or heat-exchange medium additive.

### EXAMPLES

### Example 1

Deoxybenzoin (18.83 g, 96 mmol) was introduced into a nitrogen-purged reactor (reactor volume ca. 300 ml) together with a five-fold molar excess of 50% sodium hydroxide (53.82 g, 480 mmol) and 53.82 g methyl tri-n-octyl/decyl ammonium chloride (Aliquat 336 from Cognis) (average molecular weight 442 g/mol). n-Decane (3.30 g) (internal standard for the GC evaluation) was then added. The mixture was heated with constant stirring (at 500 r.p.m.) to a reaction temperature of 45° C. 14.16 g (=120 mol-%, based on deoxybenzoin) isopropyl bromide were added at 45° C., thus starting the reaction time. The total quantity of reaction mixture amounted to 150 g which about half-filled the reactor. The conversion was determined by GC. Result: conversion after 90 mins.: >99%.

### Example 2

Deoxybenzoin (18.83 g, 96 mmol) was introduced into a nitrogen-purged reactor (reactor volume ca. 300 ml) together with a five-fold molar excess of 50% sodium hydroxide (53.82 g, 480 mmol) and methyl tert.butyl ether (MTBE, 53.82 g). n-Decane (3.30 g) (internal standard for the GC evaluation) was then added. The mixture was heated with constant stirring (at 500 r.p.m.) to a reaction temperature of 45° C. 14.16 g (=120 mol-%, based on deoxybenzoin) isopropyl bromide were added at 45° C., thus starting the reaction time. The total quantity of reaction mixture amounted to 150 g which about half-filled the reactor. The conversion was determined by GC. Result: conversion after 90 mins.: >0%.

1-15. (canceled)

16. A compound liquid at ambient temperature selected from the group consisting of tetra-alkyl ammonium salts, benzyl trialkyl ammonium salts, tetra-alkyl phosphonium salts and benzyl trialkyl phosphonium salts, provided that at least two alkyl groups are aliphatic moities having chain lengths of 8 to 10 carbon atoms and further provided that the alkyl groups are not all identical.

17. A compound or mixture of compounds liquid at ambient temperature corresponding to formula (I):
[NR¹R²R³R⁴]+[X]— (I)
wherein the substituent R¹ is a benzyl group or an alkyl group containing 1 to 4 carbon atoms, the substituents R² to R⁴ are each independently alkyl groups containing 8 to 10 carbon atoms, and X is selected from the group consisting of halide anion and alkyl sulfate anion with the formula [R⁵SO₄]⁻, wherein R⁵ is an alkyl group containing 1 to 12 carbon atoms, provided that the substituents R² to R⁴ are not all identical.

18. A compound or mixture of compounds liquid at ambient temperature corresponding to formula (Ia):
[NCH₃R²R³R⁴]+[X]— (I-a)
wherein the substituents R² to R⁴ are each independently alkyl groups containing 8 to 10 carbon atoms, X is selected from the group consisting of halide anion and alkyl sulfate anion with the formula [R⁵SO₄]⁻, wherein R⁵ is an alkyl group containing 1 to 12 carbon atoms, provided that the substituents R² to R⁴ are not all identical.

19. The mixture of compounds of claim 18, wherein X is a chloride anion and the mixture of compounds (I-a) has an average molecular weight of 420 to 440.

20. The mixture of the compounds of claim 18, wherein X is an anion selected from the group consisting of 2-ethyl hexyl sulfate, octyl sulfate and decyl sulfate.

21. A composition comprising
(a) at least 85% by weight based on the composition as a whole, of one or more compounds corresponding to formula (I):
[NR¹R²R³R⁴]+[X]— (I)
wherein the substituent R¹ is a benzyl group or an alkyl group containing 1 to 4 carbon atoms, the substituents R² to R⁴ are each independently alkyl groups containing 8 to 10 carbon atoms, and X is selected from the group consisting of halide anion and alkyl sulfate anion with the formula [R⁵SO₄]⁻, wherein R⁵ is an alkyl group containing 1 to 12 carbon atoms, and
(b) 0.01 to 10% by weight water.

22. A composition comprising
(a) at least 85% by weight based on the composition as a whole, of one or more compounds corresponding to formula (I-a):
[NCH₃R²R³R⁴]+[X]— (I-a)
wherein the substituents R² to R⁴ are each independently alkyl groups containing 8 to 10 carbon atoms, X is selected from the group consisting of halide anion and alkyl sulfate anion with the formula [R⁵SO₄]⁻, wherein R⁵ is an alkyl group containing 1 to 12 carbon atoms, and
(b) 0.01 to 5% by weight water, provided that the quantity of water in the composition is not greater than the maximum solubility of water in the compounds (Ia) at 30° C. and further provided that, if the substituents R² to R⁴ are identical, said compounds of formula (I-a) comprise at least two different compounds.

23. A method of solvating a substance comprising dissolving the substance in the compound of claim 16.

24. A method of solvating a substance comprising dissolving the substance in the compound of claim 17.

25. A method of solvating a substance comprising dissolving the substance in a composition comprising a solvent and a compound of claim 16 as a solvent additive.

26. A method of solvating a substance comprising dissolving the substance in a solvent plus the compound of claim 17 as a solvent additive.

27. A method of catalyzing a reaction comprising adding the compound of claim 16 as a phase transfer catalyst.

28. A method of catalyzing a reaction comprising adding the compound of claim 17 as a phase transfer catalyst.

29. A method of extracting a substance comprising using the compound of claim 16 as the extracting solvent.

30. A method of extracting a substance comprising using the compound of claim 17 as the extracting solvent.

31. A method of heat exchange comprising using the compound of claim 16 as the heat exchange medium.

32. A method of heat exchange comprising using the compound of claim 17 as the heat exchange medium.

33. A chemical reaction comprising the compound of claim 16.

34. A chemical reaction comprising the compound of claim 17.

35. A separation process comprising using the compound of claim 16 as a solvent or solvent additive.

36. A separation process comprising using the compound of claim 17 as a solvent or solvent additive.

37. A heat-exchange apparatus comprising the compound of claim 16 as a heat-exchange medium or heat-exchange medium additive.

38. A heat-exchange apparatus comprising the compound of claim 17 as a heat-exchange medium or heat-exchange medium additive.
